(19) United States
(12) Patent Application Publication
(10) Pub. No.: US 2009/0202571 A1
(43) Pub. Date: Aug. 13, 2009

(54) Bioreductively-activated prodrugs

(75) Inventors: Peter David Davis, Oxford (GB); Matthew Alexander Naylor, Middlesex (GB); Peter Thomson, Middlesex (GB); Steven Albert Everett, Middlesex (GB); Michael Richard Lacey Stratford, Middlesex (GB); Peter Wardman, Middlesex (GB)

Correspondence Address: HAMILTON, BROOK, SMITH & REYNOLDS, P.C., 530 VIRGINIA ROAD, P.O. BOX 9133, CONCORD, MA, 01742-9133, US

(73) Assignees: HOPNIC LABORATORY CO., LTD., SABAE-SHI (JP); DAISO CO.,LTD., NISHI-KU, OSAKA (JP)

(21) Appl. No.: 11/663,495

(22) PCT Filed: Sep. 26, 2005
(86) PCT No.: PCT/GB05/03676

§ 371 Date: May 29, 2007

(30) Foreign Application Priority Data
Sep. 24, 2004 (GB) 0421296.5

(51) Int. Cl. A61K 39/395; C07H 17/04; A61K 31/7028; A61K 38/44; A61K 31/7052; A61P 35/00

(52) U.S. Cl. 424/178.1; 536/17.6; 514/27; 424/94.4

(57) ABSTRACT

A compound of formula (1), or a pharmaceutically acceptable salt thereof, wherein: —Ar is a substituted heteroaryl group bearing at least one nitro or azido group or is a benzoquinone, naphthoquinone or fused heterocyloquinone; -R1 is hydrogen, optionally substituted alkyl, optionally substituted alkenyl, optionally substituted alkynyl, optionally substituted cycloalkyl, optionally substituted heterocycloalkyl, optionally substituted aryl or optionally substituted heteroaryl; -R2 is a glycoside, OH, optionally substituted alkyl, optionally substituted alkoxy, C₂-C₈ alkenyl, C₁-C₈ hydroxyalkyl, optionally substituted arylamino, optionally substituted aryl C₁-C₄ alkylamino or hydroxyalkylamino; and -R3 and R4 are each independently H or halo.

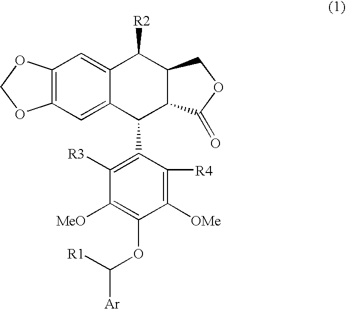

This invention relates to compounds useful in the treatment of cell proliferation disorders. More particularly the invention relates to a series of compounds that are activated under hypoxic conditions.

Many drugs used in conventional cancer chemotherapy are toxic to growing cancer cells but lack complete specificity. Thus other normal tissues are affected and ensuing side effects limit the dose that can be administered. Therefore the exposure of the cancerous tumour to the compound, and in turn the effectiveness of the therapy, is limited. There is a need for drugs that target the tumour more selectively.

Many solid tumours exhibit regions of hypoxia (low oxygen tension). Inadequate blood supply to the central regions of the tumour results in hypoxia that can be chronic or acute. This hypoxia represents a challenge to effective therapy by radiation or by conventional chemotherapy since hypoxic regions are often more resistant to these modalities. It has been suggested, however, that tumour hypoxia can be used to target tumours for drug action (Kennedy, Cancer Res. 1980, 40, 2356-2360). One particular method of using the hypoxic regions of tumours for drug targeting is the selective activation of prodrugs under conditions of low oxygen tension. A concept has been advanced whereby the activity of a cytotoxic compound can be masked by a trigger moiety which, under hypoxic conditions, mediates fragmentation of the masked cytotoxic compound into the active cytotoxic agent (Denny, Lancet Oncol 2000, 1, 25-9). Compounds attempting to utilize this concept typically consist of the trigger moiety attached, often via a linker moiety, to a cytotoxic moiety (the effector).

Hypoxia is also a feature of the rheumatoid arthritic joint (Rothschild Semin Arthritis Rheum 1982, 12, 11-31). Cell proliferation is also a feature of the arthritic joint. Systemic antiproliferative drugs (for example methotrexate) are used in the therapy of rheumatoid arthritis but are limited by side effects. Psoriatic lesions are also characterized by cell proliferation and hypoxia (Dvorak Int Arch Allergy Immunol. 1995, 107, 233-5.

A number of hypoxic trigger moieties have been disclosed including nitrobenzenes, nitronaphthalenes, nitroimidazoles, nitrofurans, nitrothiophenes, nitropyrroles, nitropyrazoles, benzoquinones, naphthoquinones, indoloquinones and azidobenzenes (for some examples see Naylor, Mini Rev. Med. Chem. 2001 1, 17-29; Tercel, J. Med. Chem. 2001, 44, 3511-3522 and Damen, Bioorg. Med. Chem. 2002, 10, 71-77).

A number of effector moieties have been utilised in the art including nitrogen mustards, phosphoramide mustards, taxanes, enediynes and indole derivatives (for some examples see Naylor, loc cit and Papot, Curr. Med. Chem. Anti Cancer Agents 2002, 2, 155-185).

Etoposide (also known as VP16) is a semisynthetic derivative of podophyllotoxin and is in current use for the treatment of solid tumours, particularly testicular and small cell lung cancers. It is thought to act as a cytotoxic agent by inducing DNA double-strand breaks via binding to the topoisomeraseII-DNA complex. Unfortunately this agent is not specific for cancer cells and also affects normal cells in a similar way, giving rise to dose-limiting side effects, especially hematologic toxicity. A close analogue of etoposide, known as teniposide, is in clinical use for the treatment of childhood acute lymphoblastic leukemia and has a similar range of side effects. A number of other etoposide analogues have been described in the art. Examples are disclosed in WO96/24602, WO99/18109, U.S. Pat. No. 5,300,500, WO2004/000859, U.S. Pat. No. 5,132,322,

Etoposide phosphate (U.S. Pat. No. 5,606,039) is a prodrug of etoposide in clinical use for the same cancer indications as etoposide. Its benefit is believed to be in its improved water solubility, lessening the potential for precipitation following dilution of the clinical formulation and during intravenous administration. Following intravenous administration it is rapidly and completely converted into etoposide in the plasma and there is no evidence of increased selectivity for the tumour over normal tissues. A number of other hydrolytically-activated etoposide prodrugs have been described, for example, Wrasidlo et al, Bioorg Med Chem Lett 2002, 12, 557-60, Told et al., J Org Chem 2002, 67, 1866-72, Schmidt and Monnerat Bioorg Med Chem 2003 11 2277 and Lange et al, Cancer Lett 2003 197, 225-30. There remains a need for etoposide analogues that are targeted to the tumour microenvironment.

It is an object of this invention to provide prodrugs that on bioreductive activation break down to release etoposide or an etoposide analogue. Thus according to one aspect of the invention we provide a compound of formula (1), or a pharmaceutically acceptable salt thereof:

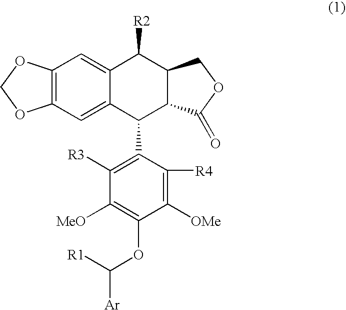

wherein:

Ar is a substituted heteroaryl group or is a benzoquinone, naphthoquinone or fused heterocyloquinone;

R1 is hydrogen, optionally substituted alkyl, optionally substituted alkenyl, optionally substituted alkynyl, optionally substituted cycloalkyl, optionally substituted heterocycloalkyl, optionally substituted aryl or optionally substituted heteroaryl;

R2 is a glycoside, OH, optionally substituted alkyl, optionally substituted alkoxy, C₂-C₈ alkenyl, C₁-C₈ hydroxyalkyl, optionally substituted arylamino, optionally substituted aryl C₁-C₄alkylamino or hydroxyalkylamino; and

R3 and R4 are each independently H or halo.

For the avoidance of doubt, the invention extends to the compounds of formula (1) in pharmaceutically acceptable solvate form.

As used herein the term “alkyl”, alone or in combinations, means a straight or branched-chain alkyl group containing from one to seven, preferably a maximum of four, carbon atoms such as methyl, ethyl, propyl, isopropyl, butyl, sec-butyl, t-butyl and pentyl. An example of an alkyl group or moiety is a linear or branched C₁-C₈ alkyl group or moiety. Typically, alkyl group or moiety is a linear or branched alkyl group or moiety containing from 1 to 6 carbon atoms, such as a C₁-C₄ or C₁-C₂ alkyl group or moiety. More preferably, an alkyl group or moiety is methyl.

As used herein, alkoxy is a said alkyl group, for example a C₁-C₄ or C₁-C₂ alkyl group, which is attached to an oxygen atom.

As used herein, a thioalkoxy group is a said alkyl group which is attached to a sulphur atom. A thioalkoxy group is also known as an alkylthio group.

Optional substituents which may be present on alkyl groups include one or more substituents selected from halogen, amino, monoalkylamino, dialkylamino, hydroxy, alkoxy, alkylthio, alkylsulphonyl, aryl, heteroaryl, acylamino, alkoxycarbonylamino, alkanoyl, acyloxy, carboxy, sulphate or phosphate groups.

A further example of an optional substituent which may be present on alkyl groups is a heterocycloalkyl group. Typically, the substituents which may be present on an alkyl group or moiety is selected from halogen, amino, mono(C₁-C₄ alkyl)amino, di(C₁-C₄ alkyl)amino, hydroxy, C₁-C₄ alkoxy, C₁-C₄ alkylthio, C₁-C₄ alkylsulphonyl, aryl, heteroaryl, acylamino, C₁-C₄ aloxycarbonylamino, C₁-C₄ alkanoyl, acyloxy, carboxy, sulphate, phosphate or heterocycloalkyl groups.

Preferably, the substituents on an alkyl group are selected from halogen, amino, mono(C₁-C₄ alkyl)amino, di(C₁-C₄ alkyl)amino, hydroxy, heterocycloalkyl and phosphate groups. Typically, alkyl groups are unsubstituted or substituted by one, two or three substitutents. Typically, said substituents which may be present on alkyl groups are themselves unsubstituted. More preferably, an alkyl group is unsubstituted.

An alkenyl group may be for example an olefinuc group containing from two to seven carbon atoms, for example ethenyl, n-propenyl, i-propenyl, n-butenyl, i-butenyl, s-butenyl and t-butenyl. Typically an alkenyl group is a C₂-C₈ alkenyl group, for example a C₂-C₆ alkenyl group. More preferably an alkenyl group is a C₂-C₄ alkenyl group. An alkenyl group typically contains only one double bond.

As used herein, an alkynyl group is a linear or branched alkynyl group. Typically an alkynyl group is a C₂-C₆, for example a C₂-C₄ alkynyl group, for example ethynyl, n-propynyl or n-butynyl. Typically, an alkynyl group contains only one triple bond. An alkynyl group may be for example an ethynyl, propynyl or butynyl group.

Preferably, an alkenyl or alkynyl group is unsubstituted or substituted by 1, 2 or 3 substituents. Preferably, the substituents on an alkenyl or alkynyl group or moiety are selected from halogen, amino, mono(C₁-C₄ alkyl)amino, di(C₁-C₄ alkyl)amino, hydroxy, C₁-C₄ alkoxy, C₁-C₄ alkylthio, (C₁-C₄ alkyl)sulphonyl groups, aryl, heteroaryl, heterocycloalkyl, acylamino, (C₁-C₄)alkoxycarbonylamino, (C₁-C₄)alkanoyl, acyloxy, carboxy, sulphate or phosphate groups. More preferably, the substituents on an alkenyl or alkynyl group or moiety are selected from halogen, amino, mono(C₁-C₂ alkyl)amino, di(C₁-C₂ alkyl)amino or hydroxy. Typically, said substituents which may be present on alkenyl or alkynyl groups are themselves unsubstituted. More preferably an alkenyl or alynyl group is unsubstituted.

The term “halogen” means fluorine, chlorine, bromine or iodine. As used herein, the term “halo” refers to a fluoro, chloro, bromo or iodo substituent. Halo is typically fluoro, chloro or bromo.

The term aryl means an unsubstituted phenyl group or a phenyl group carrying one or more, preferably one to three, substituents examples of which are halogen, optionally substituted alkyl, hydroxy, nitro, azido, cyano, amino and alkoxy. Typically an aryl group or moiety is an unsubstituted phenyl group or a phenyl group substituted with 1, 2 or 3 unsubstituted substituents selected from halogen, C₁-C₆ alkyl, hydroxy, nitro, azido, cyano, amino, C₁-C₄ haloalkyl, C₁-C₄ alkoxy and C₁-C₄ haloalkoxy.

Preferably, an aryl group is an unsubstituted phenyl group or a phenyl group substituted with 1, 2 or 3 unsubstituted substituents selected from halogen, C₁-C₆ alkyl, hydroxy, amino, C₁-C₄haloalkyl, C₁-C₄ alkoxy and C₁-C₄ haloalkoxy. More preferably, an aryl group is a phenyl group which is unsubstituted or substituted with 1, 2, or 3 unsubstituted substituents selected from halogen, C₁-C₂ alkyl, C₁-C₂ haloalkyl, C₁-C₂ alkoxy and C₁-C₂ haloalkoxy substituents.

As used herein, a haloalkyl or haloalkoxy group is a said alkyl or alkoxy group, substituted by one or more said halogen atoms. Typically, a haloalkyl or haloalkoxy group is substituted by 1, 2 or 3 said halogen atoms. Preferred haloalkyl and haloalkoxy groups include perhaloalkyl and perhaloalkoxy groups such as —CZ₃ and —OCZ₃ wherein Z is said halogen atom, for example chlorine or fluorine. Particularly preferred haloalkyl groups are —CF₃ and —CCl₃. Particularly preferred haloalkoxy groups are —OCF₃ and —OCCl₃.

The term heteroaryl is defined herein as a mono- or fused bi-cyclic aromatic group containing one to four heteroatoms selected in any combination from N, S or O atoms. A heteroaryl group is typically a 5- to 10-membered ring, such as a 5- or 6-membered ring, containing at least one heteroatom, for example 1, 2, or 3 heteroatoms chosen from N, S or O atoms. Examples of heteroaryl groups include pyridyl, pyrimidyl, furyl, thienyl, pyrrolyl, pyrazolyl, indolyl, benzofuryl, benzothienyl, benzothiazolyl, oxazolyl, isoxazolyl, thiazolyl, isothiazolyl, imidazolyl, triazolyl, quinolyl and isoquinolyl groups. A preferred example of a heteroaryl group is a thienyl group. Typically a heteroaryl group is an unsubstituted hetoraryl group or a heteroaryl group substituted with 1, 2 or 3 unsubstituted substituents selected from halogen, C₁-C₆ alkyl, hydroxy, nitro, azido, cyano, amino, C₁-C₄ haloalkyl, C₁-C₄ alkoxy and C₁-C₄ haloalkoxy, mono(C₁-C₄ alkyl)amino and di(C₁-C₄ alkyl)amino substituents. A heteroaryl group can carry one or more, preferably one to three, substituents examples of which are halogen, optionally substituted alkyl, hydroxy, nitro, azido, cyano, amino and alkoxy.

Preferably, a heteroaryl group is an unsubstituted hetoraryl group or a heteroaryl group substituted with 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy substituents.

More preferably, a heteroaryl group is unsubstituted or substituted with 1, 2, or 3 unsubstituted substituents selected from halogen, C₁-C₂ alkyl, C₁-C₂ haloalkyl, C₁-C₂ alkoxy and C₁-C₂ haloalkoxy substituents. It is further preferred that a heteroaryl group carries, where appropriate, in addition to the specified nitro or azido group, 0, 1 or 2 further unsubstituted substituents selected from halogen, C₁-C₆ alkyl, hydroxy, amino, C₁-C₄haloakyl, C₁-C₄ alkoxy and C₁-C₄ haloalkoxy, mono(C₁-C₄ alkyl)amino and di(C₁-C₄ alkyl)amino substituents. More preferably, these substituents are selected from unsubstituted C₁-C₂ alkyl substituents.

In a further embodiment, a heteroaryl group is preferably an unsubstituted heteroaryl group or a heteroaryl group substituted by one nitro group.

A heterocycloalkyl ring is typically a non-aromatic, saturated or unsaturated C₃₋₁₀ carbocyclic ring in which one or more, for example, 1, 2 or 3, of the carbon atoms are replaced by a heteroatom selected from N, O or S. Saturated heterocycloalkyl groups are preferred. Typically a heterocycloalkyl ring is a 5- to 6-membered heterocycloalkyl ring. The term heterocycloalkyl ring includes heterocycloalkyl groups containing 3-6 carbon atoms and one or two oxygen, sulphur or nitrogen atoms. Particular examples of such groups include azetidinyl, pyrrolidinyl, piperidinyl, homopiperidinyl, piperazinyl, homopiperazinyl, morpholinyl or thiomorpholinyl groups.

Substituents which may be present on a heterocycloalkyl ring include one or more groups selected from optionally substituted alkyl, halogen, oxo, hydroxy, alkoxy, alkylthio, amino, alkylamino, dialkylamino, carboxy, alkoxycarbonyl, aminocarbonyl, alkyl aminocarbonyl, dialkylaminocarbonyl, alkylsulphonyl, aminosulphonyl, acylamino, alkoxycarbonylamino, alkanoyl, acyloxy, sulphate, phosphate and alkylphosphate. Typically a heterocycloalkyl ring is an unsubstituted heterocycloalkyl group or a heterocycloalkyl group substituted with 1, 2 or 3 unsubstituted substituents selected from C₁-C₆ alkyl, C₁-C₄ haloalkyl, C₁-C₄ haloalkoxy, halogen, oxo, hydroxy, C₁-C₄ alkoxy, C₁-C₄ alkylthio, amino, mono(C₁-C₄)alkylamino, di(C₁-C₄)alkylamino, carboxy, (C₁-C₄)alkoxycarbonyl, aminocarbonyl, (C₁-C₄)alkylaminocarbonyl, di(C₁-C₄)alkylaminocarbonyl, (C₁-C₄)alkylsulphonyl, aminosulphonyl, acylamino, (C₁-C₄)alkoxycarbonylamino, (C₁-C₄)alkanoyl, acyloxy, sulphate, phosphate and (C₁-C₄)alkylphosphate.

Preferably, a heterocycloalkyl ring is an unsubstituted heterocycloalkyl group or a heterocycloalkyl group substituted with 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy. More preferably, a heterocycloalkyl ring is unsubstituted or substituted with 1, 2, or 3 unsubstituted substituents selected from halogen, C₁-C₂ alkyl, C₁-C₂ haloalkyl, C₁-C₂ alkoxy and C₁-C₂ haloalkoxy substituents.

A cycloalkyl group is typically a non-aromatic, saturated or unsaturated carbocyclic ring containing 3-10 carbon atoms such as, for example, cyclopropyl, cyclobutyl, cyclopentyl or cyclohexyl. Typically a cycloalkyl group is saturated. Typically a cycloalkyl group is a 5- or 6-membered cycloalkyl group. Substituents which may be present on a cycloalkyl group include one or more groups selected from optionally substituted alkyl, halogen, oxo, hydroxy, alkoxy, alkylthio, amino, alkylamino, dialkylamino, carboxy, alkoxycarbonyl, aminocarbonyl, alkylaminocarbonyl, dialkylaminocarbonyl, alkylsulphonyl, aminosulphonyl, acylamino, alkoxycarbonylamino, alkanoyl, acyloxy, sulphate, phosphate and alkylphosphate. Typically a cycloalkyl group ring is an unsubstituted cycloalkyl group or a cycloalkyl group substituted with 1, 2, or 3 unsubstituted substituents selected from C₁-C₆ alkyl, C₁-C₄ haloalkyl, C₁-C₄ haloalkoxy, halogen, oxo, hydroxy, C₁-C₄ alkoxy, C₁-C₄ alkylthio, amino, mono(C₁-C₄)alkylamino, di(C₁-C₄)alkylamino, carboxy, (C₁-C₄)alkoxycarbonyl, aminocarbonyl, (C₁-C₄)alkylaminocarbonyl, di(C₁-C₄)alkylaminocarbonyl, (C₁-C₄)alkylsulphonyl, aminosulphonyl, acylamino, (C₁-C₄)alkoxycarbonylamino, (C₁-C₄)alkanoyl, acyloxy, sulphate, phosphate and (C₁-C₄)alkylphosphate.

Preferably, a cycloalkyl group is unsubstituted or substituted with 1, 2, or 3 unsubstituted substituents selected from halogen, C₁-C₆ alkyl, hydroxy, amino, C₁-C₄ haloalkyl, C₁-C₄ alkoxy and C₁-C₄ haloalkoxy. More preferably, a cycloalkyl group is unsubstituted or substituted with 1, 2, or 3 unsubstituted substituents selected from halogen, C₁-C₂ alkyl, C₁-C₂ haloalkyl, C₁-C₂ alkoxy and C₁-C₂ haloalkoxy substituents.

For the avoidance of doubt, a fused heterocycloquinone group is a benzoquinone group fused to an heteroaryl or heterocycloalkyl ring, as defined above. Typically, a fused heterocycloquinone is a benzoquinone group fused to a 5- to 6-membered heteroaryl group or to a 5- to 6-membered heterocycloalkyl ring. Preferably, a fused heterocycloquinone is a benzoquinone group fused to a 5- to 6-membered heteroaryl group, for example a pyrrolyl group. An example of a fused heterocycloquinone group is a indole-4,7-dione-3yl group.

Typically, the naphthoquinone or fused heterocycloquinone group is unsubstituted or substituted by one or more, for example, 1, 2, 3 or 4 substituents. Typically, the benzoquinone group is unsubstituted or substituted by one or more, for example, 1, 2 or 3 substituents. Preferably, the benzoquinone, naphthoquinone or fused heterocycloquinone group is unsubstituted or substituted by 1, 2 or 3 substituents.

Typical substituents which may be present on the benzoquinone, naphthoquinone or the fused heterocycloquinone group include C₁-C₆ alkyl, C₁-C₄ haloalkyl, C₁—C haloalkoxy, halogen, hydroxy, C₁-C₄ alkoxy, C₁-C₄ alkylthio, amino, mono(C₁-C₄)alkylamino, di(C₁-C₄)alkylamino, heterocycloalkyl, cycloalkyl, aryl or heteroaryl. Preferred substituents are C₁-C₄ alkyl, C₁-C₄ haloalkyl, C₁-C₆ haloalkoxy, hydroxy, C₁-C₄ alkoxy and C₁-C₄ alkylthio. More preferred substituents are C₁-C₂ alkyl, C₁-C₂ haloalkyl, C₁-C₂ haloalkoxy, C₁-C₂ alkoxy and C₁-C₂ alkylthio. Typically the substituents are themselves unsubstituted.

For the avoidance of doubt, a heterocyclic group is typically a heteroaryl, heterocycloalkyl or fused heterocycloquinone group.

For the avoidance of doubt, a glycoside is an organic compound that yields a sugar and one or more nonsugar substances on hydrolysis.

Typically, in the compound of formula (1), Ar is a substituted aryl or 5- to 10-membered heteroaryl group bearing at least one nitro or azido group, or is a benzoquinone, naphthoquinone or fused heterocycloquinone. Typically, when Ar is a substituted aryl or 5- to 10-membered heteroaryl group bearing at least one nitro or azido group, it carries one substituent selected from a nitro or azido group and 0, 1 or 2 further unsubstituted substituents chosen from halogen, C₁-C₆ alkyl, hydroxy, ammo, C₁-C₄ alkyamino, C₁-C₄ dialkyamino, C₁-C₄ haloalkyl, C₁-C₄ alkoxy and C₁-C₄ haloalkoxy substituents. Preferably, said further substituents are chosen from halogen, unsubstituted C₁-C₄ alkyl, hydroxy, and C₁-C₄ dialkyamino substituents. More preferably, said substituents are unsubstituted C₁-C₂ alkyl substituents. Typically, when Ar is a substituted aryl or 5- to 10-membered heteroaryl group bearing at least one nitro or azido group, it is a phenyl or a 5- to 6-membered heteroaryl group carrying one substituent selected from a nitro or azido group, and 0, 1 or 2 said further substituents More preferably, when Ar is a substituted aryl or 5- to 10-membered heteroaryl group bearing at least one nitro or azido substituent, said group carries only one substituent which substituent is chosen from a nitro or azido group. Preferably, said substituent is a nitro group.

More typically, when Ar is a substituted 5- to 10-membered heteroaryl group bearing at least one nitro or azido group, Ar is a 5- or 6-membered heteroaryl group, for example a furanyl, imidazolyl or thienyl group, substituted by only one substituent which substituent is a nitro substituent. Particularly useful values of the moiety Ar include nitroimidazole groups, for example 2-nitroimidazol-5-yl and nitrothiophene groups, for example 5-nitrothien-2-yl. Further particularly useful examples of the moiety Ar include nitrofuranyl groups, for example 5-nitrofuran-2-yl.

Typically, when Ar is a benzoquinone, naphthoquinone or fused heterocycloquinone it is a 1,4-benzoquinone, a 1,4-naphthoquinone or an indole-4,7-dione. More typically when Ar is a benzoquinone, naphthoquinone or fused heterocycloquinone it is a 1,4-benzoquinon-2-yl, a 1,4-naphthoquinon-2-yl or an indole-4,7-dione-3-yl group. When Ar is a benzoquinone, naphthoquinone or fused heterocycloquinone such groups may be unsubstituted or have 1, 2, 3 or 4 substituents. Such substituents may be independently selected from alkyl, alkoxy, thioalkoxy, amino, alkylamino, dialkylamino, heterocycloalkyl, cycloalkyl, aryl or heteroaryl.

Preferably the group Ar in formula (1) will have a one electron reduction potential at pH7 of between −200 to −550 mV, more preferably −250 to −500 mV. One electron reduction potentials, E(1), can be obtained from literature sources or measured by a number of methods known in the art. For example E(1) can be measured by pulse radiolysis by measuring the equilibrium constant for the electron transfer between the radical anions of the compound under study and an appropriate reference standard for example a viologen or quinone compound (Meisel, J Phys Chem 1975, 79, 1503-9).

Typically, in the compound of formula (1), R1 is hydrogen, unsubstituted C₁-C₆ alkyl, a phenyl group which is unsubstituted or substituted with 1, 2 or 3 unsubstituted substituents selected from halogen, C₁-C₆ alkyl, hydroxy, amino, C₁-C₄ haloalkyl, C₁-C₄ alkoxy and C₁-C₄haloalkoxy or a heteroaryl group which is unsubstituted or substituted with 1, 2 or 3 unsubstituted substituents selected from halogen, C₁-C₆ alkyl, hydroxy, amino, C₁-C₄ haloalkyl, C₁-C₄ alkoxy and C₁-C₄ haloalkoxy substituents.

Preferably, in the compound of formula (1), R1 is hydrogen, unsubstituted C₁-C₄ alkyl, a phenyl group which is unsubstituted or substituted with 1, 2 or 3 unsubstituted substituents selected from halogen, C₁-C₄ alkyl, hydroxy, amino, C₁-C₂ haloalkyl, C₁-C₂ alkoxy and C₁-C₂ haloalkoxy.

A particularly useful group of compounds are those of formula (1) in which R1 is an alkyl group.

More preferably, in the compound of formula (1), R1 is hydrogen or unsubstituted C₁-C₂ alkyl.

Typically in the compound of formula (1), R2 is a glycoside, OH, optionally substituted alkyl, optionally substituted alkoxy, C₂-C₈ alkenyl, C₁-C₈ hydroxyalkyl, optionally substituted arylamino, optionally substituted aryl C₁-C₄alkylamino or hydroxyalkylamino. More typically, when R2 is a glycoside it is a group of formula (2) in which R5 is a C₁₋₂alkyl group or a heterocyclic group and R6 is hydroxy or dimethylamino. Preferably when R2 is a group of formula (2) R5 is methyl or 2-thienyl and R6 is hydroxy.

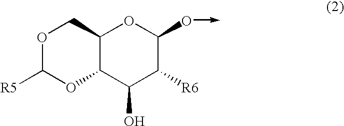

In a preferred embodiment, Ar is either:

(a) a heteroaryl group bearing at least one nitro or azido group and 0, 1 or 2 further unsubstituted substituents selected from halogen, C₁-C₆ alkyl, hydroxy, amino, C₁-C₄haloalkyl, C₁-C₄ alkoxy and C₁-C₄ haloalkoxy, mono(C₁-C₄ alkyl)amino and di(C₁-C₄ alkyl)amino substituents; or

(b) a benzoquinone group which is unsubstituted or substituted by 1, 2 or 3 unsubstituted substituents or a naphthoquinone or fused heterocyloquinone group which is unsubstituted or substituted by 1, 2, 3 or 4 unsubstituted substituents, said unsubstituted substituents being selected from C₁-C₆ alkyl, C₁-C₄ haloalkyl, C₁-C₆ haloalkoxy, hydroxy, C₁-C₄ alkoxy and C₁-C₄ alkylthio substituents. When Ar is as defined above in (a), said further unsubstituted substituents are selected from C₁-C₂ alkyl, C₁-C₂ haloalkyl, C₁-C₂ alkoxy and C₁-C₂ haloalkoxy substituents.

When Ar is as defined above in (a) it is preferably a 5- to 6-membered heteroaryl group carrying one substituent selected from a nitro or azido group and 0, 1 or 2 said further unsubstituted substituents. More preferably, when Ar is as defined above in (a), it is a 5- to 6-membered heteroaryl group bearing at least one nitro or azido substituent and 0 or 1 said further unsubstituted substituents. More preferably, when Ar is as defined above in (a), it is a 5- to 6-membered heteroaryl group, (for example thienyl) bearing only one substituent which substituent is a nitro group. In one embodiment, Ar is not 1-methyl-2-nitro-imidazol-5-yl. In a further embodiment, Ar is not a substituted imidazolyl group.

Preferably, when Ar is as defined above in (b), said substituents are selected from unsubstituted C₁-C₂ alkyl, C₁-C₂ haloalkyl, C₁-C₂ haloalkoxy, C₁-C₂ alkoxy and C₁-C₂ alkylthio groups. Preferably, when R1 is as defined above in (b), it is a benzoquinone, naphthoquinone or a fused heterocycloquinone group wherein a benzoquinone group is fused to a 5- to 6-membered heteroaryl group, which is unsubstituted or substituted by 1, 2, or 3 said unsubstituted substituents.

In a preferred embodiment, Ar is a group as defined above in (a).

In a preferred embodiment, R1 is hydrogen or an unsubstituted C₁-C₆ alkyl, C—C₂-C₆ alkenyl, C₂-C₆ alkynyl, C₃-C₁₀ cycloalkyl, 5- to 6-membered heterocycloalkyl, phenyl or 5- to 10-membered heteroaryl group. R1 is preferably hydrogen or an unsubstituted C₁-C₄ alkyl group. More preferably R1 is hydrogen or an unsubstituted C₁-C₂ alkyl group.

R3 is typically H or fluoro, chloro or bromo. Preferably, R3 is H.

R4 is typically H or fluoro, chloro or bromo. Preferably, R4 is H.

In a preferred embodiment, R2 is a glycoside, OH, or an unsubstituted C₁-C₆ alkyl, C₁-C₆ alkoxy, C₂-C₆ alkenyl, C₁-C₆ hydroxyalkyl, phenylamino, phenyl C₁-C₆ alkylamino or hydroxy C₁-C₆ alkylamino group.

In a preferred embodiment, R2 is not: [tetrahydro-2-methyl-3aH-[1,3]dioxolo[4,5-c]pyran-6,7-diol-4-yl]-CH₂—.

In a preferred embodiment, R2 is not: [tetrahydro-2-methyl-3aH-[1,3]dioxolo[4,5-c]pyran-6,7-diol-4-yl]-O—.

In a preferred embodiment, R2 is not: [hexahydro-2-methylpyrano[3,2-d][1,3]dioxine-7,8-diol-6-yl]-CH₂—.

In a more preferred embodiment, R2 is not [tetrahydro-2-methyl-3aH-[1,3]dioxolo[4,5-c]pyran-6,7-diol-4-yl]-CH₂—, [tetrahydro-2-methyl-3aH-[1,3]dioxolo[4,5-c]pyran-6,7-diol-4-yl]-O— or [hexahydro-2-methylpyrano[3,2-d][1,3]dioxine-7,8-diol-6-yl]-CH₂—.

Typically, when R2 is [hexahydro-2-methylpyrano[3,2-d][1,3]dioxine-7,8-diol-6-yl]-O—, Ar is not 1-methyl-2-nitro-imidazol-5-yl. Preferably, when R2 is [hexahydro-2-methylpyrano[3,2-d][1,3]dioxine-7,8-diol-6-yl]-O—, Ar is not a substituted imidazolyl group. More preferably, when R2 is [hexahydro-2-methylpyrano[3,2-d][1,3]dioxine-7,8-diol-6-yl]-O—, Ar is a thienyl group bearing at least one nitro or azido group.

Preferably, R2 is a glycoside of formula (2):

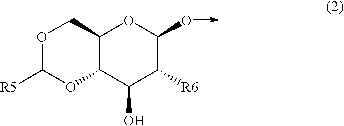

in which:

R5 is C₁-C₂ alkyl group or a heterocyclic group; and

R6 is hydroxy or dimethylamino.

R5 is typically an unsubstituted C₁-C₂ alkyl group, a 5- to 6-membered heteroaryl group which is substituted with 0, 1 or 2 unsubstituted substituents selected from halogen, C₁-C₂ alkyl, C₁-C₂ haloalkyl, C₁-C₂ alkoxy and C₁-C₂ haloalkoxy substituents or a 5- to 6-membered heterocycloalkyl group which is substituted with 0, 1 or 2 unsubstituted substituents selected from halogen, C₁-C₂ alkyl, C₁-C₂ haloalkyl, C₁-C₂ alkoxy and C₁-C₂ haloaloxy substituents. Preferably, R5 is a C₁-C₂ alkyl or a 5- to 6-membered heteroaryl group which is substituted by 0 or 1 unsubstituted substituents selected from halogen, C₁-C₂ alkyl, C₁-C₂ haloalkyl, C₁-C₂ alkoxy and C₁-C₂ haloalkoxy substituents. More preferably, R5 is an unsubstituted C₁-C₂ alkyl group or an unsubstituted 5- to 6-membered heteroaryl group, for example thienyl.

R6 is preferably a hydroxy group.

In a preferred embodiment, when R5 is methyl and R6 is hydroxy, Ar is not 1-methyl-2-nitro-imidazol-5-yl. In a more preferred embodiment, when R5 is methyl and R6 is hydroxy, Ar is not a substituted imidazolyl group. In a further preferred embodiment, when R5 is methyl and R6 is hydroxy, Ar is a thienyl group bearing at least one nitro or azido group.

In a more preferred embodiment, in the compound of formula (1):

Ar is either:

(a) a heteroaryl group bearing at least one nitro or azido group and 0, 1 or 2 further unsubstituted substituents selected from halogen, C₁-C₆ alkyl, hydroxy, amino, C₁-C₄ haloalkyl, C₁-C₄ alkoxy and C₁-C₄ haloalkoxy, mono(C₁-C₄ alkyl)amino and di(C₁-C₄ alkyl)amino substituents; or

(b) a benzoquinone or naphthoquinone group which is unsubstituted or substituted by 1, 2 or 3 unsubstituted substituents or a fused heterocyloquinone group which is unsubstituted or substituted by 1, 2, 3 or 4 unsubstituted substituents, said unsubstituted substituents being selected from C₁-C₆ alkyl, C₁-C₄ haloalkyl, C₁-C₆ haloalkoxy, hydroxy, C₁-C₄ alkoxy and C₁-C₄ alkylthio substituents;

R1 is hydrogen or an unsubstituted C₁-C₆ alkyl, C—C₂-C₆ alkenyl, C₂-C₆ alkynyl, C₃-C₁₀ cycloalkyl, 5- to 6-membered heterocycloalkyl, phenyl or 5- to 10-membered heteroaryl group;

R3 is H or fluoro, chloro or bromo;

R4 is H or fluoro, chloro or bromo; and

R2 is a glycoside, OH, or an unsubstituted C₁-C₆ alkyl, C₁-C₆ alkoxy, C₂-C₆ alkenyl, C₁-C₆ hydroxyalkyl, phenylamino, phenyl C₁-C₆ alkylamino or hydroxy C₁-C₆ alkylamino group. Preferably in this embodiment R2 is not [tetrahydro-2-methyl-3aH-[1,3]dioxolo[4,5-c]pyran-6,7-diol-4-yl]-CH₂—, [tetrahydro-2-methyl-3aH-[1,3]dioxolo[4,5-c]pyran-6,7-diol-4-yl]-O— or [hexahydro-2-methylpyrano[3,2-d][1,3]dioxine-7,8-diol-6-yl]-CH₂—, and when R2 is [hexahydro-2-methylpyrano[3,2-d][1,3]dioxine-7,8-diol-6-yl]-O—, Ar is not 1-methyl-2-nitro-imidazol-5-yl.

In a further preferred embodiment, in the compound of formula (1):

Ar is a 5- to 6-membered heteroaryl group bearing only one substituent which substituent is a nitro group;

R1 is hydrogen or an unsubstituted C₁-C₂ alkyl group;
R3 is H;
R4 is H; and

R2 is a glycoside of formula (2) in which:
R5 is an unsubstituted C₁-C₂ alkyl group or an unsubstituted 5- to 6-membered heteroaryl group and
R6 is a hydroxy group.

Most preferably, the compound of formula (1) is selected from:
9-[(4,6-O-Ethylidene-β-D-glucopyranosyl)oxy]-5,8,8a,9-tetrahydro-5-(4-(1-(5-nitrothien-2-yl)ethoxy)-3,5-dimethoxyphenyl)furo[3′,4′:6,7]naphtha[2,3-d]-1,3-dioxol-6(5aH)-one;
9-[(4,6-O-Ethylidene-β-D-glucopyranosyl)oxy]-5,8,8a,9-tetrahydro-5-(4-(5-nitrothien-2-yl)methoxy)-3,5-dimethoxyphenyl)furo[3′,4′:6,7]naphtha[2,3-d]-1,3-dioxol-6(5aH)-one;
9-[(4,6-O-(Thien-2-ylmethylidene-β-D-glucopyranosyl)oxy]-5,8,8a,9-tetrahydro-5-(4-(5-nitrothien-2-yl)methoxy)-3,5-dimethoxyphenyl)furo[3′,4′:6,7]naphtha[2,3-d]-1,3-dioxol-6(5aH)-one; and
9-[(4,6-O-Thien-2-ylmethylidene-β-D-glucopyranosyl)oxy]-5,8,8a,9-tetrahydro-5-(4-(1-(5-nitrothien-2-yl)ethoxy)-3,5-dimethoxyphenyl)furo[3′,4′:6,7]naphtha[2,3-d]-1,3-dioxol-6(5aH)-one.

Where one or more functional groups in compounds of formula (1) are sufficiently basic or acidic the formation of salts is possible. Suitable salts include pharmaceutically acceptable salts for example acid addition salts including hydrochlorides, hydrobromides, phosphates, sulphates, hydrogen sulphates, alkylsulphonates, arylsulphonates, acetates, benzoates, citrates, maleates, fumarates, succinates, lactates and tartrates, salts derived from inorganic bases including alkali metal salts such as sodium or potassium salts, alkaline earth metal salts such as magnesium or calcium salts, and salts derived from organic amines such as morpholine, piperidine or dimethylamine salts.

Those skilled in the art will recognise that compounds of formula (1) may exist as stereoisomers and/or geometrical isomers and accordingly the present invention includes all such isomers which have anticancer activity and mixtures thereof.

It is a further object of this invention to provide methods for the preparation of compounds of formula (1).

Compounds of formula (1) may be prepared by a number of processes as generally described below and more specifically in the Examples hereinafter. In the following process description, the symbols Ar, R1, R2, R3 and R4 when used in the formulae depicted are to be understood to represent those groups described above in relation to formula (1) unless otherwise indicated. In the schemes described below it may be necessary to employ protecting groups that are then removed during the final stages of the synthesis. The appropriate use of such protecting groups and processes for their removal will be readily apparent to those skilled in the art.

Compounds of formula (1) can be prepared by Mitsunobu reaction of an alcohol of formula (3) with an epipodophyllotoxin analogue of formula (4) in a solvent such as an ether solvent, for example tetrahydrofuran, diethyl ether or dioxan or in a solvent such as an aromatic hydrocarbon for example benzene or toluene or in a solvent such as an aprotic solvent for example dimethylformamide, in the presence of a phosphine for example triphenylphosphine or tri-n-butylphosphine and in the presence of an azo compound such as diethylazodicarboxylate, diisopropylazodicarboxylate or 1,1′-(azodicarbonyl)dipiperidine at a temperature from about 0° C. to about the reflux temperature of the solvent, conveniently at room temperature.

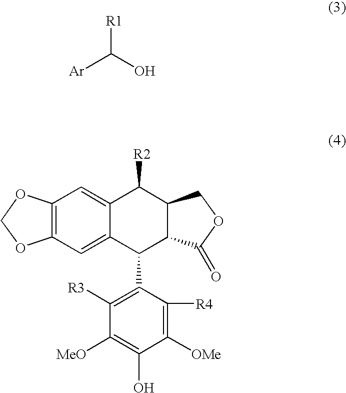

Phenols of formula (4) are either known in the art or can be prepared by standard methods apparent to one skilled in the art.

Alcohols of formula (3) are either known or can be prepared by standard methods apparent to one skilled in the art. Such methods include treatment of an aldehyde or ketone of formula (5) with a reducing agent, for example a borohydride reducing agent such as sodium borohydride in a solvent such as an alcoholic solvent for example methanol at a temperature between about −20° C. to room temperature, preferably around 0° C. Such methods also include the treatment of an aldehyde of formula (6) with an organometallic compound of formula (7) in which M represents a metal, metal halide or dialkylmetal, for example, Li, ZnBr, MgBr or MgI or dialkylaluminium in a solvent such as an ether solvent, for example tetrahydrofuran or diethyl ether or in an aromatic solvent for example benzene or toluene at a temperature of between about −78° C. to about the reflux temperature of the solvent, preferably from about 0° C. to room temperature. Where Ar is a substituted heteroaryl group bearing at least one nitro group such methods also include the aromatic electrophilic nitration of the appropriate heteroaryl substrate with an appropriate nitrating agent at a temperature of between about −78° C. and room temperature. Appropriate nitrating agents are, for example, nitric acid in a solvent such as an acid anhydride for example acetic anhydride or in a solvent such as an acid for example sulphuric acid or acetic acid; nitronium tetrafluoroborate in a solvent such as an ether solvent, for example tetrahydrofuran or diethyl ether or in a solvent such as acetonitrile or glacial acetic acid or in a solvent such as a chlorinated solvent for example dichloromethane or dinitrogen tetroxide in a solvent such as an ether solvent, for example tetrahydrofuran or diethyl ether or in a solvent such as acetonitrile or glacial acetic acid or in a solvent such as a chlorinated solvent for example dichloromethane or in an aromatic solvent for example benzene or toluene.

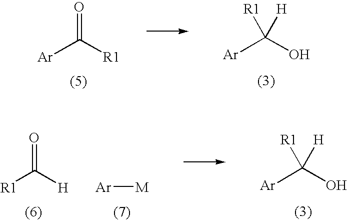

Compounds of formula (1) can also be synthesized from other compounds of formula (1) by the application of standard methods, including substitution reactions, functional group transformations, bond-forming reactions and cyclisations known in the art. For example a compound of formula (1) containing an ester group may be hydrolysed under acidic or basic catalysis to give the corresponding carboxylic acid. A compound of formula (1) containing a carboxylic acid can be treated with ammonia, a monoalkylamine or a dialkylamine under standard coupling conditions to give an amide.

Preparation of a compound of formula (1) as a single enantiomer or, where appropriate, diastereomer may be effected by synthesis from an enantiomerically pure starting material or intermediate or by resolution of the final product in a conventional manner.

The compounds of the invention may be administered as a sole therapy or in combination with other treatments. For the treatment of solid tumours compounds of the invention may be administered in combination with radiotherapy or in combination with other anti-tumour substances for example those selected from mitotic inhibitors, for example vinblastine, vincristine, vinorelbine, paclitaxel and docetaxel; alkylating agents, for example cisplatin, carboplatin, oxaliplatin, nitrogen mustard, melphalan, chlorambucil, busulphan and cyclophosphamide; antimetabolites, for example 5-fluorouracil, cytosine arabinoside, gemcitabine, capecitabine, methotrexate and hydroxyurea; intercalating agents for example adriamycin and bleomycin; enzymes, for example aspariginase; topoisomerase inhibitors for example etoposide, teniposide, topotecan and irinotecan; thymidylate synthase inhibitors for example raltitrexed; biological response modifiers for example interferon; antibodies for example edrecolomab, cetuximab, bevacizumab and trastuzumab; receptor tyrosine kinase inhibitors for example gefitinib, imatinib and erlotinib; and anti-hormones for example tamoxifen, anastrazole, exemestane and letrozole. Such combination treatment may involve simultaneous or sequential application of the individual components of the treatment.

For the prophylaxis and treatment of disease the compounds according to the invention may be administered as pharmaceutical compositions selected with regard to the intended route of administration and standard pharmaceutical practice. Such pharmaceutical compositions may take a form suitable for oral, buccal, nasal, topical, rectal or parenteral administration and may be prepared in a conventional manner using conventional excipients. For example for oral administration the pharmaceutical compositions may take the form of tablets or capsules. For nasal administration or administration by inhalation the compounds may be conveniently delivered as a powder or in solution. Topical administration may be as an ointment or cream and rectal administration may be as a suppository. For parenteral injection (including intravenous, subcutaneous, intramuscular, intravascular or infusion) the composition may take the form of, for example, a sterile solution, suspension or emulsion.

The dose of a compound of the invention required for the prophylaxis or treatment of a particular condition will vary depending on the compound chosen, the route of administration, the form and severity of the condition and whether the compound is to be administered alone or in combination with another drug. Thus the precise dose will be determined by the administering physician but in general daily dosages may be in the range 0.001 to 100 mg/kg preferably 0.1 to 10 mg/kg. Typically, daily dosage levels are from 0.05 mg to 2 g, for example from 5 mg to 1 g.

The compounds of the present invention are therapeutically useful in treating, preventing, ameliorating or reducing incidence of a proliferative disorder. Typically, the proliferative disorder is a hypoxic disorder. A hypoxic disorder is typically a disorder in which diseased cells are present in a hypoxic environment. Examples of the disorders that can be treated, prevented, ameliorated or disorders whose incidence can be reduced, include cancer, rheumatoid arthritis, psoriatic lesions, diabetic retinopathy or wet age-related macular degeneration.

Typically, the disorder is cancer. Preferably the cancer is a hypoxic cancer. A hypoxic cancer is, of course, a cancer wherein cancerous cells are in a hypoxic environment. Most preferably, the cancer is a solid tumour or leukaemia. Typically the leukaemia is leukaemia involving the spleen or bone marrow or is childhood acute lymphoblastic leukaemia. Typically, the solid tumour is testicular tumour or a small cell lung tumour.

According to a further aspect of the invention there is provided a compound of formula (1), or a pharmaceutically acceptable salt or solvate thereof, for use in a method of treatment of the human or animal body by therapy. In particular the present invention provides a method of ameliorating or reducing the incidence of a proliferative disorder as defined above in a patient, which method comprises administering to said patient an effective amount of a compound of formula (1) or a pharmaceutically acceptable salt thereof.

A further feature of the present invention is a compound of formula (1), or a pharmaceutically acceptable salt or solvate thereof, for use as a medicament. In particular, the present invention provides a compound of formula (1), or a pharmaceutically acceptable salt thereof, for the treatment of the human or animal body.

According to a further aspect of the invention there is provided the use of a compound of formula (1), or a pharmaceutically acceptable salt or solvate thereof, in the manufacture of a medicament for use in the therapy of a warm-blooded animal, for example a human, suffering from a proliferative disease for example cancer. In particular, the present invention provides the use of a compound of formula (1), or a pharmaceutically acceptable salt thereof, in the manufacture of a medicament for use in the treatment of the human or animal body, for the prevention or treatment of a said proliferative disorder.

A number of enzymes are capable of reducing aryl and heteroaryl nitro groups. Strategies that increase the activity of such enzymes within solid tumours can therefore increase further the activity of prodrugs dependent on nitro reduction. Similarly a number of enzymes are capable of reducing quinones and indoloquinones and therefore similar strategies are possible to increase the effectiveness of drugs requiring activation by quinone reduction. Such strategies include linking such enzymes to a tumour-targeting antibody, administering such enzyme antibody conjugates to a host with a solid tumour then, after the conjugate has localised to the tumour, administering the prodrug. This approach is known as Antibody Directed Enzyme Prodrug Therapy (ADEPT). Alternatively the gene encoding for the enzyme might be delivered selectively and/or expressed selectively, in the tumour before administration of the prodrug. This approach is known as Gene Directed Enzyme Prodrug Therapy (GDEPT). When the gene is delivered by a viral vector the approach is sometimes known as Virus Directed Enzyme Prodrug Therapy (VDEPT).

Anlezark has disclosed nitroreductases and their use in an ADEPT strategy. Prodrugs for use in this strategy were also disclosed (U.S. Pat. No. 5,633,158 and U.S. Pat. No. 5,977,065). In WO 00 047725 Anlezark provides further disclosures of nitroreductase enzymes and their use in GDEPT strategies. Denny (WO 00 064864) has disclosed nitroaryl and nitroheteroaryl prodrugs for use in a GDEPT strategy. The use of quinone-reducing enzymes in ADEPT, GDEPT and MDEPT (Macromolecule Directed Enzyme Prodrug Therapy) is discussed in Skelly et al. Mini Rev Med. Chem. 2001, 1, 293-306.

Thus it is a further object of this invention to provide the use of compounds of formula (1) in combination with a reductase, an antibody-reductase conjugate, a macromolecule-reductase conjugate or DNA encoding a reductase gene, in a method of treatment for the human body. Thus, the present invention provides a method of ameliorating or reducing the incidence of a said proliferative disorder in a patient, which method comprises administering to said patient an effective amount of
(a) a compound of formula (1), or a pharmaceutically acceptable salt thereof; and
(b) a reductase, an anti-body reductase conjugate, a macromolecule-reductase conjugate or DNA encoding a reductase gene.

Further, the present invention provides a product containing
(a) a compound of formula (1), or a pharmaceutically acceptable salt thereof; and
(b) a reductase, an anti-body reductase conjugate, a macromolecule-reductase conjugate or DNA encoding a reductase gene
   for simultaneous, separate or sequential use in the treatment of a proliferative condition.

The ability of compounds of the invention to release etoposide or an etoposide analogue selectively under hypoxic conditions can be assessed by using, for example, one or more of the procedures set out below:

### Radiolysis

In the hypoxic environments of solid tumours, prodrugs can be reduced by processes that are inhibited in the normoxic environments of normal tissues. Radiolysis demonstrates the ability of bioreductively-activated prodrugs to release the active drug after reduction. Compounds were dissolved in an isopropanol/water mixture (50:50) at a concentration of 5011M or below. Solutions, in gas-tight syringes, were saturated with nitrous oxide before irradiation in a ⁶⁰Co source at a dose rate of 3.9Gy min⁻¹ (as determined by Fricke dosimetry: H. Fricke and E. J. Hart, “Chemical Dosimetry” in Radiation Dosimetry Vol. 2 (F. H. Attrix and W. C. Roesch. Eds.), pp 167-239. Academic Press New York, 1966). Solutions were analysed for released drug by HPLC. In this test example compounds of the invention produced cytotoxic etoposide analogues efficiently with radiation chemical yields (G-value) as shown in Table 1.

TABLE 1
Release of drugs from prodrugs by radiolysis

| Compound | Drug | |
|---|---|---|
| of Example | released | G (μmol · J⁻¹) |
| 1 | etoposide | 0.32 |
| 2 | etoposide | 0.40 |
| 3 | teniposide | 0.57 |

### Drug Release by Human Cytochrome P450 Reductase

Cytochrome p450 reductase is widely expressed in human tumours as well as in a range of normal tissues and is one of a number of enzymes that can catalyse bioreduction. This assay shows the ability of prodrugs to fragment into active drugs catalysed by cytochrome p450 selectively under conditions of low oxygen. Compounds were dissolved in DMSO to a concentration of 625 μM and 20 μL added to a mixture of 50 mmol dm⁻³ potassium phosphate buffer at pH 7.4 (2.4 mL), NADPH (20 μL of a 10 mM solution) and 60 μL of Supersomal™ human P450 reductase (Gentest; Catalogue number P244) or 25 μL of bactosomal human P450 reductase (Cypex; Catalogue number Cyp004) and incubated at 37° C. For experiments under nitrogen the mixture was degassed with nitrogen for 20 minutes prior to compound addition and overgassed with nitrogen during the incubation. Samples (100 μl) were taken at regular intervals and added to an equivalent volume of acetonitrile, then mixed and centrifuged at 14, 300 RPM for 2 min prior to product analysis by HPLC. In this assay the compound of Example 1 produced etoposide at a rate of 0.83 nmol min⁻¹ mg protein⁻¹ under anoxia but no drug production could be detected under air.

### Metabolism in Tumour Homogenates

Useful bioreductive prodrugs can be shown to release the active drug selectively under conditions of low oxygen in the presence of tumour homogenate in this assay. Freshly-excised CaNT tumours (approximately 0.5 to 1 g) were homogenised in 15 ml of ice-cold 50 mmol din⁻³ potassium phosphate buffer at pH 7.4. The homogenates were centrifuged at 1000 RPM for 10 min and the supernatants stored on ice. The metabolism of 5 μmol dm⁻³ prodrug in air and N₂ was performed with 0.5 ml tumour homogenate (˜3 mg of protein by Bradford assay) with 100 μmol dm⁻³ NADPH in 50 mmol dm⁻³ potassium phosphate buffer at pH 7.4 incubated at 37° C. Samples (60 μl) were taken at regular intervals and added to an equivalent volume of acetonitrile, then mixed and centrifuged at 14, 300 RPM for 2 min prior to product analysis by HPLC. In this assay the compound of Example 1 produced etoposide at 17 μmol/min/mg protein under nitrogen but no etoposide production was detected under air.

### Metabolism in Whole Cells

The ability of the prodrugs to release the drug in hypoxic whole cell cultures can be assessed by the following assay. A549 cells (ca. 4×10⁵ per well) were incubated in 6-well plates at 37° C. overnight in air or 0.2% oxygen before addition of test compound (0.75 ml, dissolved in DMSO and diluted with cell culture medium to final concentration of 5 μM). Incubation was continued and samples removed at intervals for analysis by HPLC. Compounds of the invention efficiently produced etoposide or etoposide analogues under 0.2% oxygen and the rate of production was much slower under air.

### Cellular Cytotoxicity

In a preferred embodiment of the invention the compounds of formula (1) will be less potent as cytotoxic agents than the corresponding etoposide compounds which are released under hypoxic conditions. The cytotoxic or cytostatic properties of compounds of formula (1) and corresponding etoposide compounds can be assessed for example, by use, for example, of this assay. The Celltiter 96® Aqᵤₑₒᵤₛ One Solution Cell Proliferation Assay kit (Promega Corporation, USA) which is a colorimetric method for determining the number of viable cells in proliferation or cytotoxicity assays was used. In this assay the MTS tetrazolium compound (Owen's Reagent) is bioreduced by viable cells into a coloured formazan product which is soluble in tissue culture medium and can be measured by recording absorbance at 490 nm with a 96 well plate reader. A549 cells were seeded in Eagles Minimum Essential Medium supplemented with 10% foetal calf serum and non-essential amino acids at 10³ cell per well on a 96 well plate and allowed to attach for 24 h. Compounds were dissolved in DMSO and diluted with cell culture medium before addition. The cells were exposed to test compound for either 6 h or 48 h. The MTS reagent was added to each well, left for 4 h, then the absorbance measured at 490 nm with a 96 well plate reader. In this assay, using the 61 h incubation period, the compound of Example 1 was without effect up to the highest concentration tested (20 μM) whereas etoposide itself had an IC50 of 7.5 μM.

### Metabolism in Liver Homogenates

Release of parent drug from bioreductive prodrugs under hypoxia can be demonstrated using liver homogenates as a source of the reductase enzymes also present in solid tumours. Metabolic stability of the compounds and unfavorable release of the drug by oxic liver can also be assessed by using this assay. Freshly-excised mouse liver (approximately 1 g) was homogenised in 15 ml of ice-cold 50 mmol dm⁻³ potassium phosphate buffer at pH 7.4. The homogenates were centrifuged at 1000 RPM for 10 min and the supernatants stored on ice. The metabolism of 5 □mol dm⁻³ prodrug in air was performed with 0.5 ml liver homogenate (˜2 mg of protein by Bradford assay) with 100 □mol dm⁻³ NADPH in 50 mmol dm⁻³ potassium phosphate buffer at pH 7.4 incubated at 37° C. Samples (60 □l) were taken at regular intervals and added to an equivalent volume of acetonitrile, then mixed and centrifuged at 14, 300 RPM for 2 min prior to product analysis by HPLC. Example compounds of the invention efficiently released cytotoxic nucleoside analogues under nitrogen (anoxic) but the release under air (oxic) was much slower.

TABLE 2
Oxygen-inhibited release of drugs from example compounds
of the invention catalysed by liver homogenate

| | | |
|---|---|---|
| | | Rate of drug release |
| Compound | Drug | (pmol/min/mg protein) |

| of Example | released | Anoxic | Oxic |
|---|---|---|---|
| 1 | etoposide | 95 | 0 |
| 2 | etoposide | 388 | 7 |

### EXAMPLE 1

### 9-[(4,6-O-Ethylidene-β-D-glucopyranosyl)oxy]-5,8,8a,9-tetrahydro-5-(4-(1-(5-nitrothien-2-yl)ethoxy)-3,5-dimethoxyphenyl)furo[3′,4′:6,7]naphtha[2,3-d]-1,3-dioxol-6(5aH)-one

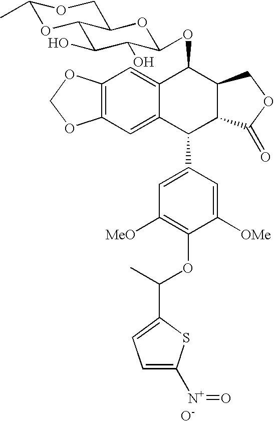

2-(1-Hydroxyethyl)-5-nitrothiophene (18 mg, 0.1 mmol) was dissolved in THF (0.5 ml) together with etoposide (76.4 mg, 0.26 mmol) and Ph₃P (70 mg, 0.26 mmol) under a nitrogen atmosphere. DEAD (50 mg, 0.29 mmol) was then added and the solution was stirred at reflux for 2 hours and then applied directly to a silica column, which was eluted with ethyl acetate/hexane (1:1). The product obtained was re-chromatographed, this time eluting with ethyl acetate to give the required compound as a pale yellow wax (15 mg, 20%). MS (m/z, %) 588 (3.6%), 382 (3.2%), 156 (27%), 141 (100%) LC-RT 6.06 minutes (TFA 50-100%). ¹H NMR (500 MHz, CDCl₃) □ 7.81 (1H, d, J=5.0, HarH), 6.94 (1H, d, J=5.0, HarH), 6.88 (1H, s, ArH), 6.60 (1H, s, ArH), 6.29 (2H, s, ArH), 6.07 (1H, s, OCH₂O), 6.04 (1H, s, OCH₂O), 5.39 (1H, bs, HarCHO), 4.97 (1H, bs, OCHO), 4.80 (1H, bs, OCHO), 4.71 (1H, d, J=5.0, ArCH), 4.66 (1H, d, J=5.0, ArCHAr), 4.46 (1H, t, J=10.0, CO₂CHH), 4.29-4.22 (2H, m, CO₂CHH, OCH), 3.78 (1H, m, OCHH), 3.63 (6H, S, OCH₃), 3.59 (1H, t, J=5.0, CHOH), 3.50 (1H, m, CHOH), 3.40 (2H, d, J=5.0, 2×OCH), 3.33 (1H, dt, J=5.0, ArCHCH), 2.91 (1H, m, CH), 1.45 (3H, d, J=5.0, CH₃), 1.33 (3H, m, CH₃) ppm.

### EXAMPLE 2

### 9-[(4,6-O-Ethylidene-β-D-glucopyranosyl)oxy]-5,8,8a,9-tetrahydro-5-(4-(5-nitrothien-2-yl)methoxy)-3,5-dimethoxyphenyl)furo[3′,4′:6,7]naphtha[2,3-d]-1,3-dioxol-6(5aH)-one

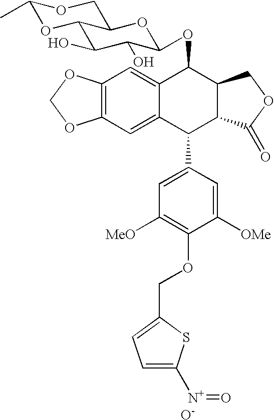

5-nitrothien-2-ylmethanol (32 mg, 0.2 mmol) was dissolved in THF (2 mL) together with triphenylphosphine (140 mg, 0.52 mmol) and etoposide (170 mg, 0.28 mmol). To this was added diethylazodicarboxylate (100 mg, 0.6 mmol) and the solution stirred at 20° C. for 18 h. The solution was evaporated to dryness and the residue purified by flash chromatography, eluting with 2% methanol/DCM to give the title compound as an off-white solid (60 mg, 41%). TLC R10.2, 2% methanol/DCM. LC-RT 6.3 min (TFA20-50%); MS m/z 588/399/382/324/2458/201/154/143. ¹H NMR (500 MHz, CDCl₃) □ 7.82 (1H, d, J=4.1, HarH), 7.02 (1H, d, J=4.1, HarH), 6.88 (1H, s, ArH), 6.59 (1H, s, ArH), 6.31 (2H, s, ArH), 6.05 (1H, d, J=1.2, OCH₂O), 6.02 (1H, d, J=1.2, OCH₂O), 5.16 (2H, s, HarCH₂O), 4.98 (1H, d, J=3.5, OCHO), 4.80 (1H, q, J=5.0, OCHO), 4.48 (2H, t, J=7.8, ArCHAr, ArCHCH), 4.29 (1H, t, J=9.8, CO₂CM), 4.23 (1H, t, J=8.7, CO₂CHH), 4.22 (1H, dd, J=6.1, 2.1, OCH), 3.77 (1H, m, J=5.3, OCHH), 3.77 (6H, s, OCH₃), 3.63 (1H, m, J=5.6, CHOH), 3.49 (1H, t, J=8.3, CHOH), 3.39 (2H, d, J=3.5, 2×OCH), 3.36 (1H, dt, J=5.3, ArCHCH), 2.92 (1H, m, CH), 2.75 (1H, bs, OH), 1.70 (1H, bs, OH), 1.44 (3H, d, J=5.0, CH₃) ppm.

### EXAMPLE 3

### 9-[(4,6-O-(Thien-2-ylmethylidene-β-D-glucopyranosyl)oxy]-5,8,8a,9-tetrahydro-5-(4-(5-nitrothien-2-yl)methoxy)-3,5-dimethoxyphenyl)furo[3′,4′:6,7]naphtha[2,3-d]-1,3-dioxol-6(5aH)-one

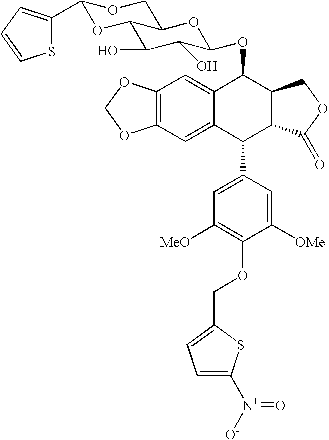

5-nitrothien-2-ylmethanol (24 mg, 0.15 mmol) was dissolved in THF (2 mL) together with triphenylphosphine (98 mg, 0.375 mmol) and teniposide (131 mg, 0.2 mmol). To this was added diethylazodicarboxylate (78 mg, 0.45 mmol) and the solution stirred at 20° C. for 18 h, evaporated to dryness and purified by flash chromatography, eluting with 2% methanol/DCM to give a pale yellow oil, which was purified further by flash chromatography, eluting with ethyl acetate to give 46 mg (38%) of the title compound as a white solid. TLC R_{f}=0.85, ethyl acetate. LC-RT 3.53 min (TFA50-100%); MS m/z 656/400/382/353/337/245/229/201/185/167/154/143. ¹H NMR (500 MHz, CDCl₃) □ 8.03 (1H, d, J=5.0, HarH), 7.55 (1H, d, J=5.0, HarH), 7.19 (2H, m, HarH), 7.05 (1H, m, HarH), 6.56 (1H, s, ArH), 6.60 (1H, s, ArH), 6.28 (2H, s, ArH), 6.05 (2H, s, OCH₂O), 5.90 (1H, s, OCHO), 5.31 (1H, bs, OH), 5.27 (1H, bs, OH), 5.10 (2H, s, HarCHO), 4.98 (1H, bs, OCHO), 4.65 (1H, d, J=5.0, OCHO), 4.59 (1H, d, J=5.0, ArCH), 4.30 (2H, d, J=5.0, ArCHAr), 4.25 (1H, d, J=10.0, CO₂CHH), 3.76 (1H, m, OCHH), 3.63 (6H, s, OCH₃), 3.40-3.33 (4H, m, 4×CH), 3.13 (1H, m, ArCHCH), 2.92 (1H, m, CH) ppm

### EXAMPLE 4

### 9-[(4,6-O— Thien-2-ylmethylidene-β-D-glucopyranosyl)oxy]-5,8,8a,9-tetrahydro-5-(4-(1-(5-nitrothien-2-yl)ethoxy)-3,5-dimethoxyphenyl)furo[3′,4′:6,7]naphtha[2,3-d]-1,3-dioxol-6(5aH)-one

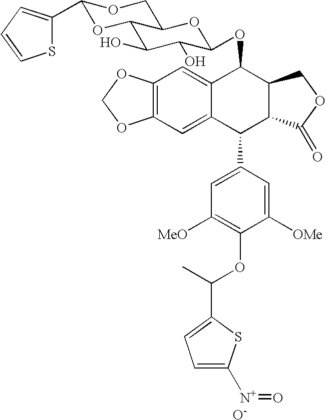

2-(1-Hydroxyethyl)-5-nitrothiophene (26 mg, 0.15 mmol) was dissolved in THF (2 mL) together with triphenylphosphine (98 mg, 0.375 mmol) and teniposide (131 mg, 0.2 mmol). To this was added diethylazodicarboxylate (78 mg, 0.45 mmol) and the solution stirred at 20° C. for 18 h, evaporated to dryness and purified by flash chromatography, eluting with 2% methanol/DCM to give a pale yellow oil, which was triturated with ether to reveal an off-white solid (23 mg, 19%); TLC R_{f}=0.20, 2% methanol/DCM; ¹H NMR (250 MHz, CDCl₃) □ 8.00 (1H, d, J=4.3, HarH), 7.55 (1H, d, J=4.3, HarH), 7.18 (1H, d, J=2.6, HarH), 7.13 (1H, d, J=2.6, HarH), 7.03 (1H, m, HarH), 6.54 (2H, s, ArH), 6.26 (2H, s, ArH), 6.04 (2H, bs, OCH₂O), 5.89 (1H, s, OCHO), 5.31 (1H, m, HarCHO), 5.28 (1H, bs, OH), 5.26 (1H, bs, OH), 4.97 (1H, m, OCHO), 4.64 (1H, d, J=7.4, OCHO), 4.58 (1H, d, J=5.2, ArCH), 4.35-4.22 (4H, m, ArCHAr, CO₂CH₂, OCH), 3.72 (2H, m, OCHH, CHOH), 3.62. (6H, s, OCH₃), 3.51 (1H, s, CHOH), 3.43 (2H, bs, 2×OCH), 3.39 (1H, s, ArCHCH), 1.52 (3H, d, J=4.7, CH₃) ppm; LC-RT 6.48 min (TFA20-50%); MS (m/z, %) 655, 400, 384, 324, 274, 185, 157, 143.

1. A compound of formula (1), or a pharmaceutically acceptable salt thereof, 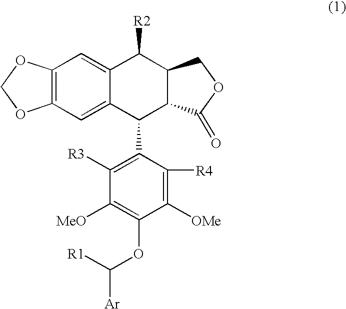
wherein:
Ar is a substituted heteroaryl group bearing at least one nitro or azido group or is a benzoquinone, naphthoquinone or fused heterocyloquinone;
R1 is hydrogen, optionally substituted alkyl, optionally substituted alkenyl, optionally substituted alkynyl, optionally substituted cycloalkyl, optionally substituted heterocycloalkyl, optionally substituted aryl or optionally substituted heteroaryl;
R2 is a glycoside, OH, optionally substituted alkyl, optionally substituted alkoxy, C₂-C₈ alkenyl, C₁-C₈ hydroxyalkyl, optionally substituted arylamino, optionally substituted aryl C₁-C₄alkylamino or hydroxyalkylamino; and
R3 and R4 are each independently H or halo.

2. A compound according to claim 1, wherein:
the alkyl, alkenyl and alkynyl groups or moieties are unsubstituted or substituted by 1, 2 or 3 unsubstituted substituents selected from halogen, amino, mono(C₁-C₄ alkyl)amino, di(C₁-C₄ alkyl)amino, hydroxy, C₁-C₄ alkoxy, C₁-C₄ alkylthio, C₁-C₄-alkylsulphonyl, aryl, heteroaryl, acylamino, C₁-C₄ alkoxycarbonylamino, C₁-C₄ alkanoyl, acyloxy, carboxy, sulphate, phosphate or heterocycloalkyl groups;
the alkenyl and alkynyl groups are unsubstituted or substituted by 1, 2 or 3 unsubstituted substituents selected from halogen, amino, mono(C₁-C₄ alkyl)amino, di(C₁-C₄ alkyl)amino, hydroxy, C₁-C₄ alkoxy, C₁-C₄ alkylthio, (C₁-C₄ alkyl)sulphonyl groups, aryl, heteroaryl, heterocycloalkyl, acylamino, (C₁-C₄)alkoxycarbonylamino, (C₁-C₄)alkanoyl, acyloxy, carboxy, sulphate or phosphate groups;
the heteroaryl and aryl groups are unsubstituted or substituted with 1, 2 or 3 unsubstituted substituents selected from halogen, C₁-C₆ alkyl, hydroxy, nitro, azido, cyano, amino, C₁-C₄ haloalkyl, C₁-C₄ alkoxy and C₁-C₄ haloalkoxy, mono(C₁-C₄ alkyl)amino and di(C₁-C₄ alkyl)amino substituents;
the heterocycloalkyl and cycloalkyl groups are unsubstituted or substituted with 1, 2 or 3 unsubstituted substituents selected from C₁-C₆ alkyl, C₁-C₄ haloalkyl, C₁-C₄ haloalkoxy, halogen, oxo, hydroxy, C₁-C₄ alkoxy, C₁-C₄ alkylthio, amino, mono(C₁-C₄)alkylamino, di(C₁-C₄)alkylamino, carboxy, (C₁-C₄)alkoxycarbonyl, aminocarbonyl, (C₁-C₄)alkylaminocarbonyl, di(C₁-C₄)alkylaminocarbonyl, (C₁-C₄)alkylsulphonyl, aminosulphonyl, acylamino, (C₁-C₄)alkoxycarbonylamino, (C₁-C₄)alkanoyl, acyloxy, sulphate, phosphate and (C₁-C₄)alkylphosphate;
the benzoquinone group is unsubstituted or substituted by 1, 2 or 3 unsubstituted substituents selected from C₁-C₆ alkyl, C₁-C₄ haloalkyl, C₁—C haloalkoxy, halogen, hydroxy, C₁-C₄ alkoxy, C₁-C₄ alkylthio, amino, C₁-C₄ alkylamino, di(C₁-C₄)alkylamino, heterocycloalkyl, cycloalkyl, aryl or heteroaryl; and
the naphthoquinone or fused heterocyloquinone group is unsubstituted or substituted by 1, 2, 3 or 4 unsubstituted substituents selected from C₁-C₆ alkyl, C₁-C₄ haloalkyl, C₁—C haloalkoxy, halogen, hydroxy, C₁-C₄ alkoxy, C₁-C₄ alkylthio, amino, C₁-C₄ alkylamino, di(C₁-C₄)alkylamino, heterocycloalkyl, cycloalkyl, aryl or heteroaryl.

3. Compound according to claim 1 wherein Ar is either
(a) a 5- to 6-membered heteroaryl group carrying one substituent selected from a nitro or azido group and 0, 1 or 2 further unsubstituted substituents selected from C₁-C₂ alkyl, C₁-C₂ haloalkyl, C₁-C₂ alkoxy and C₁-C₂ haloalkoxy substituents; or
(b) a benzoquinone, naphthoquinone or a fused heterocycloquinone group wherein a benzoquinone group is fused to a 5- to 6-membered heteroaryl group, which is unsubstituted or substituted by 1, 2, or 3 unsubstituted substituents selected from unsubstituted C₁-C₂ alkyl, C₁-C₂ haloalkyl, C₁-C₂ haloalkoxy, C₁-C₂ alkoxy and C₁-C₂ alkylthio groups.

4. Compound according to claim 3 wherein Ar is as defined in (a).

5. Compound according to claim 1 wherein R1 is hydrogen or an unsubstituted C₁-C₄ alkyl group.

6. Compound according to claim 1 wherein R3 is H.

7. Compound according to claim 1 wherein R4 is H.

8. Compound according to claim 1 wherein R2 is a glycoside, OH, or an unsubstituted C₁-C₆ alkyl, C₁-C₆ alkoxy, C₂-C₆ alkenyl, C₁-C₆ hydroxyalkyl, phenylamino, phenyl C₁-C₆ alkylamino or hydroxy C₁-C₆ alkylamino group.

9. Compound according to claim 1 wherein R2 is a group of formula (2): 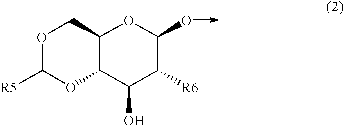
in which:
R5 is C₁-C₂ alkyl group or a heterocyclic group; and
R6 is hydroxy or dimethylamino.

10. Compound according to claim 9 wherein:
R5 is an unsubstituted C₁-C₂ alkyl group or an unsubstituted 5- to 6-membered heteroaryl group; and
R6 is a hydroxy group.

11. Compound according to claim 1 wherein when R2 is [hexahydro-2-methylpyrano[3,2-d][1,3]dioxine-7,8-diol-6-yl]-O—, Ar is not 1-methyl-2-nitro-imidazol-5-yl.

12. Compound according to claim 1 wherein Ar is not a substituted imidazolyl group.

13. Compound according to claim 1 selected from:
9-[(4,6-O-Ethylidene-β-D-glucopyranosyl)oxy]-5,8,8a,9-tetrahydro-5-(4-(1-(5-nitrothien-2-yl)ethoxy)-3,5-dimethoxyphenyl)furo[3′,4′:6,7]naphtha[2,3-d]-1,3-dioxol-6(5aH)-one;
9-[(4,6-O-Ethylidene-β-D-glucopyranosyl)oxy]-5,8,8a,9-tetrahydro-5-(4-(5-nitrothien-2-yl)methoxy)-3,5-dimethoxyphenyl)furo[3′,4′:6,7]naphtha[2,3-d]-1,3-dioxol-6(5aH)-one;
9-[(4,6-O-(Thien-2-ylmethylidene-β-D-glucopyranosyl)oxy]-5,8,8a,9-tetrahydro-5-(4-(5-nitrothien-2-yl)methoxy)-3,5-dimethoxyphenyl)furo[3′,4′:6,7]naphtha[2,3-d]-1,3-dioxol-6(5aH)-one;
9-[(4,6-O— Thien-2-ylmethylidene-β-D-glucopyranosyl)oxy]-5,8,8a,9-tetrahydro-5-(4-(1-(5-nitrothien-2-yl)ethoxy)-3,5-dimethoxyphenyl)furo[3′,4′:6,7]naphtha[2,3-d]-1,3-dioxol-6(5aH)-one, or a pharmaceutically acceptable salt thereof.

14. A pharmaceutical composition comprising a compound according to claim 1, or a pharmaceutically acceptable salt thereof, and a pharmaceutically acceptable carrier or diluent.

15-19. (canceled)

20. A method of ameliorating or reducing the incidence of a proliferative disorder in a patient, which method comprises administering to said patient an effective amount of a compound as defined in claim 1, or a pharmaceutically acceptable salt thereof.

21. A method of ameliorating or reducing the incidence of a proliferative disorder in a patient, which method comprises administering to said patient an effective amount of
(a) a compound as defined in claim 1, or a pharmaceutically acceptable salt thereof; and
(b) a reductase, an anti-body reductase conjugate, a macromolecule-reductase conjugate or DNA encoding a reductase gene.

22. (canceled)

23. Method according to claim 20, wherein the proliferative disorder is cancer, rheumatoid arthritis, psoriatic lesions, diabetic retinopathy or wet age-related macular degeneration.

24. Method according to claim 20, wherein the proliferative disorder is a hypoxic disorder.

25. Method according to claim 20, wherein the proliferative disorder is a solid tumour or leukaemia.
